(19) United States
(12) Patent Application Publication
(10) Pub. No.: US 2024/0148236 A1
(43) Pub. Date: May 9, 2024

(54) MEDICAL SUPPORT DEVICE, ENDOSCOPE APPARATUS, MEDICAL SUPPORT METHOD, AND PROGRAM

(71) Applicant: FUJIFILM Corporation, Tokyo (JP)

(72) Inventor: Nobuyuki Torisawa, Kanagawa (JP)

(73) Assignee: FUJIFILM Corporation, Tokyo (JP)

(21) Appl. No.: 18/490,783

(22) Filed: Oct. 20, 2023

(30) Foreign Application Priority Data
Nov. 4, 2022 (JP) 2022-177610

(51) Int. Cl. A61B 1/00; A61B 1/018; A61B 1/05; A61B 18/14; G06V 20/52

(52) U.S. Cl. CPC A61B 1/0005; A61B 1/000094; A61B 1/018; A61B 1/05; A61B 18/1492; G06V 20/52; A61B 2018/00601; A61B 2018/141; A61B 2018/144; G06V 2201/031

(57) ABSTRACT

A medical support device includes a processor used for an endoscope apparatus including an endoscope that images a luminal organ and a treatment tool that is energized in a state of being in contact with a treatment target part included in the luminal organ to cut the treatment target part. The processor is configured to output permission and non-permission information indicating whether or not the energization is permitted. Whether or not the energization is permitted is determined on the basis of a captured image that has been obtained by imaging the luminal organ with the endoscope and that includes the treatment tool.

### CROSS-REFERENCE TO RELATED APPLICATIONS

This application claims priority under 35 USC 119 from Japanese Patent Application No. 2022-177610 filed on Nov. 4, 2022, the disclosure of which is incorporated by reference herein.

### BACKGROUND

### 1. Technical Field

The technology of the present disclosure relates to a medical support device, an endoscope apparatus, a medical support method, and a program.

### 2. Related Art

JP2000-262537A discloses a treatment tool for an endoscope apparatus in which a conductive knife wire is exposed from a distal end part of an insertion portion inserted into a body through an endoscope apparatus and a high-frequency current is applied to the knife wire to incise a diseased part. The treatment tool for an endoscope apparatus disclosed in JP2000-262537A is provided with an insulating portion that is insulated to any length from a distal end of an exposed portion of the knife wire exposed from the distal end part of the insertion portion.

WO2021/140616A discloses a treatment system comprising a treatment tool, an imaging device, and a control device including a processor that controls an operation of the treatment tool. In the treatment system disclosed in WO2021/140616A, the treatment tool incises a biological tissue by applying treatment energy to the biological tissue from an end effector according to supplied power. The imaging device generates a captured image obtained by imaging a state in which the treatment energy is applied to the biological tissue from the end effector. The processor acquires the captured image, determines whether or not the incision of the biological tissue has been completed on the basis of the captured image, and stops the supply of power to the treatment tool in a case in which it is determined that the incision of the biological tissue has been completed.

### SUMMARY

One embodiment according to the technology of the present disclosure provides a medical support device, an endoscope apparatus, a medical support method, and a program that can energize a treatment tool in a safe state.

According to a first aspect of the technology of the present disclosure, there is provided a medical support device comprising: a processor used for an endoscope apparatus including a camera (an endoscope) that images a luminal organ and a treatment tool that is energized in a state of being in contact with a treatment target part included in the luminal organ to cut the treatment target part. The processor is configured to output permission and non-permission information indicating whether or not the energization is permitted, and whether or not the energization is permitted is determined on the basis of a captured image that has been obtained by imaging the luminal organ with the endoscope and that includes the treatment tool.

According to a second aspect of the technology of the present disclosure, in the medical support device according to the first aspect, the treatment tool may have a conductor that comes into contact with the treatment target part, and the conductor may be included in the captured image.

According to a third aspect of the technology of the present disclosure, in the medical support device according to the second aspect, the processor may be configured to output the permission and non-permission information on the basis of an image in which the conductor and an insulating region insulating the conductor are included as the captured image.

According to a fourth aspect of the technology of the present disclosure, in the medical support device according to the first aspect, the treatment tool may have an insulating region, and the energization may be permitted in a case in which the insulating region is included in the captured image.

According to a fifth aspect of the technology of the present disclosure, in the medical support device according to the first aspect, the treatment tool may have an insulating region and a conductor that comes into contact with the treatment target part, and the energization may be permitted in a case in which the insulating region and the conductor are included in the captured image.

According to a sixth aspect of the technology of the present disclosure, in the medical support device according to any one of the third to fifth aspects, an indicator that is capable of specifying the insulating region may be given to the insulating region.

According to a seventh aspect of the technology of the present disclosure, in the medical support device according to any one of the first to fifth aspects, a process based on the captured image may be performed as a process required to determine whether or not the energization is permitted in a case in which a first instruction has been received by a receiving device, and the permission and non-permission information may be output in a case in which a second instruction has been received by the receiving device.

According to an eighth aspect of the technology of the present disclosure, in the medical support device according to any one of the first to sixth aspects, an indicator may be given to the treatment tool, and the processor may be configured to output the permission and non-permission information on the basis of an image in which the indicator is included as the captured image.

According to a ninth aspect of the technology of the present disclosure, in the medical support device according to any one of the first to seventh aspects, whether or not the energization is permitted may be determined on the basis of a size and/or position of the treatment tool included in the captured image.

According to a tenth aspect of the technology of the present disclosure, in the medical support device according to any one of the first to ninth aspects, the treatment tool may have a conductor that comes into contact with the treatment target part, and whether or not the energization is permitted may be determined on the basis of a size and/or position of the conductor included in the captured image.

According to an eleventh aspect of the technology of the present disclosure, in the medical support device according to any one of the first to ninth aspects, the treatment tool may have an insulating region, and whether or not the energization is permitted may be determined on the basis of a size and/or position of the insulating region included in the captured image.

According to a twelfth aspect of the technology of the present disclosure, in the medical support device according to any one of the first to eleventh aspects, the endoscope apparatus may have an insertion portion that is inserted into the luminal organ, the endoscope may be provided in the insertion portion, the insertion portion may have an opening and an insertion passage that leads to the opening, the insertion passage may have a conductive region, the treatment tool may protrude from the opening through the insertion passage, an insulating region may be provided in the treatment tool, and whether or not the energization is permitted may be determined according to an aspect in which the treatment tool is included in the captured image.

According to a thirteenth aspect of the technology of the present disclosure, in the medical support device according to any one of the first to twelfth aspects, the treatment tool may include a wire.

According to a fourteenth aspect of the technology of the present disclosure, in the medical support device according to the thirteenth aspect, the treatment tool may be a papillotomy knife including the wire.

According to a fifteenth aspect of the technology of the present disclosure, in the medical support device according to the thirteenth aspect, the treatment tool may be a snare including the wire.

According to a sixteenth aspect of the technology of the present disclosure, in the medical support device according to any one of the thirteenth to fifteenth aspects, the processor may be configured to: set an energized state in which the energization is performed on the wire on condition that a state of the wire included in the captured image is a treatable state in which the treatment target part is capable of being treated by performing the energization; and set a non-energized state in which the energization is not performed on the wire on condition that the state of the wire included in the captured image is not the treatable state.

According to a seventeenth aspect of the technology of the present disclosure, in the medical support device according to any one of the first to sixteenth aspects, whether or not the energization is permitted may be determined according to an aspect in which the treatment tool is included in the captured image.

According to an eighteenth aspect of the technology of the present disclosure, in the medical support device according to any one of the first to seventeenth aspects, the treatment tool may have a conductor that comes into contact with the treatment target part, an insulating region, and at least one indicator, and the conductor, the insulating region, and/or the at least one indicator may be included in the captured image.

According to a nineteenth aspect of the technology of the present disclosure, in the medical support device according to any one of the first to eighteenth aspects, an object treated by the treatment tool may be a duodenal papilla.

According to a twentieth aspect of the technology of the present disclosure, in the medical support device according to any one of the first to nineteenth aspects, the output of the permission and non-permission information may be displaying the permission and non-permission information on a screen.

According to a twenty-first aspect of the technology of the present disclosure, in the medical support device according to any one of the first to the twentieth aspects, the captured image may be displayed on a screen.

According to a twenty-second aspect of the technology of the present disclosure, there is provided an endoscope apparatus comprising: the medical support device according to any one of the first to twenty-first aspects; and a camera (an endoscope).

According to a twenty-third aspect of the technology of the present disclosure, there is provided a medical support method used for an endoscope apparatus including a camera (an endoscope) that images a luminal organ and a treatment tool that is energized in a state of being in contact with a treatment target part included in the luminal organ to cut the treatment target part. The medical support method comprises outputting permission and non-permission information indicating whether or not the energization is permitted. Whether or not the energization is permitted is determined on the basis of a captured image that has been obtained by imaging the luminal organ with the endoscope and that includes the treatment tool.

According to a twenty-fourth aspect of the technology of the present disclosure, there is provided a program causing a computer used for an endoscope apparatus including a camera (an endoscope) that images a luminal organ and a treatment tool that is energized in a state of being in contact with a treatment target part included in the luminal organ to cut the treatment target part to perform a process comprising outputting permission and non-permission information indicating whether or not the energization is permitted. Whether or not the energization is permitted is determined on the basis of a captured image that has been obtained by imaging the luminal organ with the endoscope and that includes the treatment tool.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which a duodenoscope system is used;

FIG. 2 is a conceptual diagram illustrating an example of an overall configuration of the duodenoscope system;

FIG. 3 is a block diagram illustrating an example of a hardware configuration of an electrical system of the duodenoscope system;

FIG. 4 is a block diagram illustrating an example of functions of main units of a processor included in an endoscope apparatus and an example of information stored in an NVM;

FIG. 5 is a conceptual diagram illustrating an example of the content of a process of a detection unit;

FIG. 6 is a conceptual diagram illustrating an example of the content of a process of a control unit;

FIG. 7 is a flowchart illustrating an example of a flow of a medical support process;

FIG. 8 is a flowchart illustrating a first modification example of the flow of the medical support process;

FIG. 9 is a flowchart illustrating a second modification example of the flow of the medical support process;

FIG. 10 is a flowchart illustrating a third modification example of the flow of the medical support process;

FIG. 11 is a conceptual diagram illustrating a modification example of the content of the process of the control unit;

FIG. 12 is a conceptual diagram illustrating a first modification example of the content of the process of the detection unit;

FIG. 13 is a conceptual diagram illustrating a second modification example of the content of the process of the detection unit; and

FIG. 14 is a conceptual diagram illustrating a third modification example of the content of the process of the detection unit.

### DETAILED DESCRIPTION

Hereinafter, examples of embodiments of a medical support device, an endoscope apparatus, a medical support method, and a program according to the technology of the present disclosure will be described with reference to the accompanying drawings.

The terms used in the following description will be described first.

CPU is an abbreviation of “central processing unit”. GPU is an abbreviation of “graphics processing unit”. RAM is an abbreviation of “random-access memory”. NVM is an abbreviation of “non-volatile memory”. EEPROM is an abbreviation of “electrically erasable programmable read-only memory”. ASIC is an abbreviation of “application-specific integrated circuit”. PLD is an abbreviation of “programmable logic device”. FPGA is an abbreviation of “field-programmable gate array”. SoC is an abbreviation of “system-on-chip”. SSD is an abbreviation of “solid-state drive”. USB is an abbreviation of “Universal Serial Bus”. HDD is an abbreviation of “hard disk drive”. EL is an abbreviation of “electroluminescence”. CMOS is an abbreviation of “complementary metal-oxide-semiconductor”. CCD is an abbreviation of “charge-coupled device”. AI is an abbreviation of “artificial intelligence”. BLI is an abbreviation of “blue light imaging”. LCI is an abbreviation of “linked color imaging”. OF is an abbreviation of “interface”.

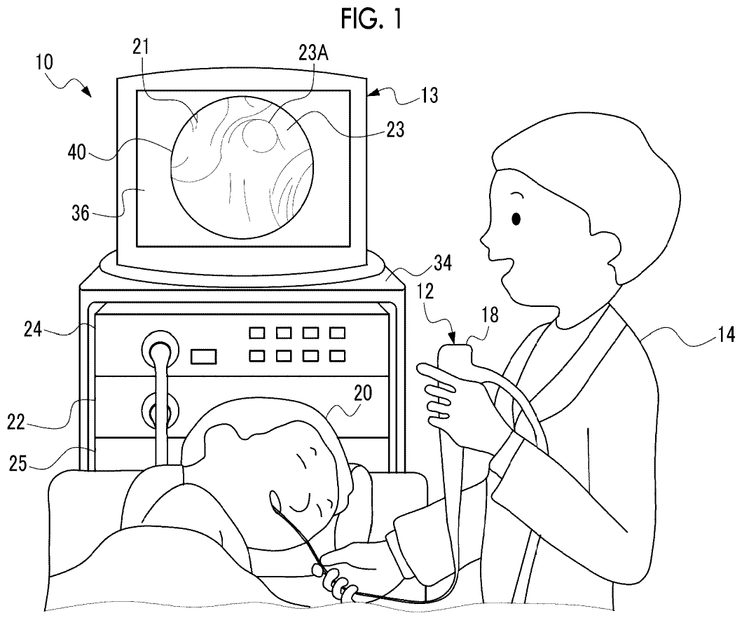

For example, as illustrated in FIG. 1, a duodenoscope system 10 comprises a duodenoscope apparatus 12 and a display device 13. The duodenoscope apparatus 12 is used by a doctor 14 in endoscopy. The duodenoscope apparatus 12 is an example of an “endoscope apparatus” according to the technology of the present disclosure.

The duodenoscope apparatus 12 is connected to a communication device (not illustrated) such that it can communicate, and information obtained by the duodenoscope apparatus 12 is transmitted to the communication device. The communication device receives the information transmitted from the duodenoscope apparatus 12 and performs a process using the received information (for example, a process of recording the information on an electronic medical record or the like).

The duodenoscope apparatus 12 comprises a duodenoscope apparatus main body 18. The duodenoscope apparatus 12 is an apparatus for performing a medical examination on a duodenum 21 included in a body of a subject 20 (for example, a patient) using the duodenoscope apparatus main body 18. The duodenum 21 is an object observed by the doctor 14.

The duodenoscope apparatus main body 18 is inserted into the body of the subject 20. The duodenoscope apparatus 12 directs the duodenoscope apparatus main body 18 inserted into the body of the subject 20 to image the duodenum 21 in the body of the subject 20 and performs various medical treatments on the duodenum 21 as needed.

The duodenoscope apparatus 12 images the inside of the body of the subject 20 to acquire an image showing an aspect of the inside of the body and outputs the image. Further, in this embodiment, the duodenoscope apparatus 12 is an endoscope apparatus having an optical imaging function that irradiates the inside of the body with light and that captures light reflected by an intestinal wall 23 of the duodenum 21. In this embodiment, the duodenum 21 is an example of a “luminal organ” according to the technology of the present disclosure.

The duodenoscope apparatus 12 comprises a control device 22, a light source device 24, and an image processing device 25. The control device 22, the light source device 24, and the image processing device 25 are installed in a wagon 34. A plurality of tables are provided in the wagon 34 along a vertical direction, and the image processing device 25, the control device 22, and the light source device 24 are installed from a lower table to an upper table. In addition, the display device 13 is installed on the uppermost table in the wagon 34.

The control device 22 controls the entire duodenoscope apparatus 12. The image processing device 25 performs various types of image processing on the image obtained by imaging the intestinal wall 23 with the duodenoscope apparatus main body 18 under the control of the control device 22.

The display device 13 displays various types of information including the image. An example of the display device 13 is a liquid-crystal display or an EL display. In addition, a tablet terminal with a display may be used instead of the display device 13 or together with the display device 13.

A screen 36 is displayed on the display device 13. A captured image 40 obtained by the duodenoscope apparatus 12 is displayed on the screen 36. The captured image 40 is an image obtained by imaging the intestinal wall 23 with the duodenoscope apparatus main body 18 in the body of the subject 20. In the example illustrated in FIG. 1, the intestinal wall 23 including a treatment target part 23A is included in the captured image 40. An example of the treatment target part 23A is a duodenal papilla. The treatment target part 23A is an example of a “treatment target part”, an “object treated by a treatment tool”, and a “duodenal papilla” according to the technology of the present disclosure.

In the following description, for convenience of explanation, an intestinal wall image is given as an example of the captured image 40. The intestinal wall image means an image obtained by imaging the intestinal wall 23. In addition, the duodenum is only an example, and any region that can be imaged by the duodenoscope apparatus 12 may be used. For example, an esophagus or a stomach is given as an example of the region that can be imaged by the duodenoscope apparatus 12. The captured image 40 is an example of a “captured image” according to the technology of the present disclosure.

A moving image configured to include the captured images 40 of a plurality of frames in time series is displayed on the screen 36. That is, the captured images 40 of a plurality of frames are displayed in time series on the screen 36 at a predetermined frame rate (for example, several tens of frames/sec). The screen 36 is an example of a “screen” according to the technology of the present disclosure.

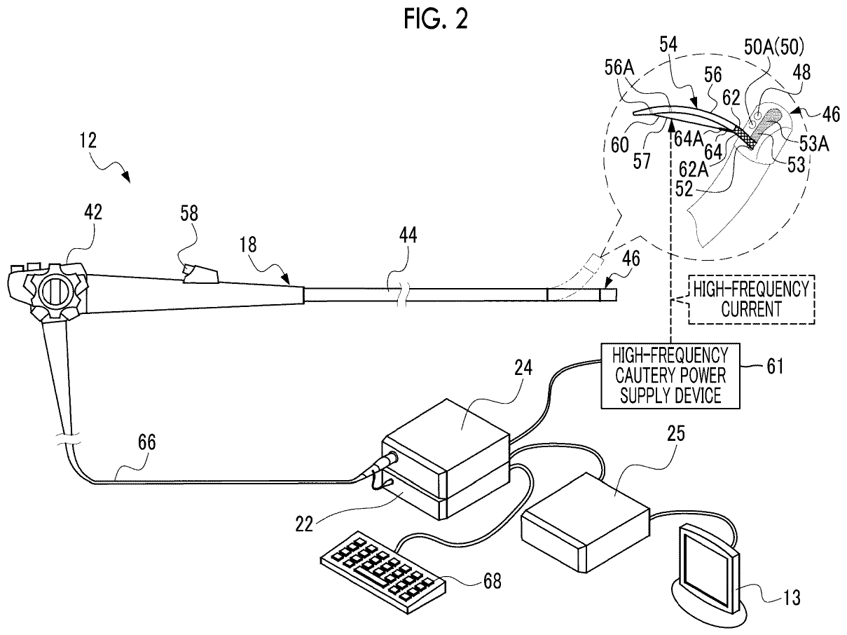

For example, as illustrated in FIG. 2, the duodenoscope apparatus main body 18 comprises an operation portion 42 and an insertion portion 44. The insertion portion 44 is partially curved by the operation of the operation portion 42. The insertion portion 44 is inserted into the duodenum 21 while being curved according to the shape of the duodenum 21 in response to the operation of the operation portion 42 by the doctor 14. The insertion portion 44 is an example of an “insertion portion” according to the technology of the present disclosure.

A distal end part 46 of the insertion portion 44 is provided with a camera 48 (an endoscope), an illumination device 50, and a treatment opening 52. The camera 48 and the illumination device 50 are provided on a side surface of the distal end part 46. That is, the duodenoscope apparatus 12 is configured as a side endoscope apparatus and enables a user to easily observe the intestinal wall of the duodenum.

The camera 48 is a device that images the inside of the body (for example, the inside of the duodenum 21) of the subject 20 and that acquires the captured image 40 as a medical image. An example of the camera 48 is a CMOS camera. However, this is only an example, and the camera 48 may be other types of cameras such as CCD cameras. The camera 48 is an example of an “endoscope” according to the technology of the present disclosure.

The illumination device 50 has an illumination window 50A. The illumination device 50 emits light through the illumination window 50A. Examples of the type of light emitted from the illumination device 50 include visible light (for example, white light) and invisible light (for example, near-infrared light). In addition, the illumination device 50 emits special light through the illumination window 50A. Examples of the special light include light for BLI and/or light for LCI. The camera 48 images the inside of the body of the subject 20 using an optical method in a state in which the illumination device 50 irradiates the inside of the body of the subject 20 with light.

The treatment opening 52 is used as a treatment tool protruding port through which a treatment tool protrudes from the distal end part 46, a suction port for sucking, for example, blood and body waste, and a delivery port for sending out a fluid. The treatment opening 52 is an example of an “opening” according to the technology of the present disclosure.

An insertion passage 53 that leads to the treatment opening 52 is formed in the insertion portion 44. The insertion passage 53 has a conductive region 53A. The conductive region 53A is a region that is made of a conductive material (for example, stainless steel).

The treatment tool protrudes from the treatment opening 52 through the insertion passage 53 in response to the operation by the doctor 14. The treatment tool is inserted into the insertion portion 44 through a treatment tool insertion opening 58. The treatment tool passes through the insertion portion 44 through the treatment tool insertion opening 58 and protrudes from the treatment opening 52 into the body of the subject 20. In the example illustrated in FIG. 2, an aspect in which, as the treatment tool, a papillotomy knife 54 protrudes from the treatment opening 52 is illustrated. The papillotomy knife 54 is only an example of the treatment tool, and other examples of the treatment tool include a catheter, a guide wire, a cannula, and a snare. In this embodiment, the papillotomy knife 54 is an example of a “treatment tool” and a “papillotomy knife” according to the technology of the present disclosure.

The papillotomy knife 54 is a treatment tool that is energized (for example, is energized by a high-frequency current) in a state of being in contact with the treatment target part 23A to cut the treatment target part 23A.

The papillotomy knife 54 comprises a cannula 56 and an incision wire 57. The cannula 56 is formed in a tubular shape. The cannula 56 is provided with a plurality of indicators 56A along a longitudinal direction. The plurality of indicators 56A are, for example, a plurality of regions having different colors and/or patterns.

The incision wire 57 is a wire that is made of a conductor 60. An example of the conductor 60 is stainless steel. The incision wire 57 is an example of a “wire” according to the technology of the present disclosure, and the conductor 60 is an example of a “conductor” according to the technology of the present disclosure.

A high-frequency cautery power supply device 61 is connected to the incision wire 57. The high-frequency cautery power supply device 61 is operated by the doctor 14 to perform energization with a high-frequency current (hereinafter, simply referred to as “energization”) on the incision wire 57 or to stop the energization.

One end of the incision wire 57 is coupled to a distal end part of the cannula 56, and the other end of the incision wire 57 extends from the treatment tool insertion opening 58 to the outside of the duodenoscope apparatus main body 18. A portion of the incision wire 57 between the one end and the other end is covered with an insulating sheath 62 together with a portion of the cannula 56. The insulating sheath 62 is made of, for example, a fluororesin. An indicator 62A is attached to the insulating sheath 62. The indicator 62A is, for example, a color and/or a pattern that can specify the insulating sheath 62. The indicator 62A is an example of an “indicator” according to the technology of the present disclosure.

The incision wire 57 has an insulating coating region 64. The insulating coating region 64 is a region in which a portion, which is close to the insulating sheath 62, in the incision wire 57 exposed from the insulating sheath 62 to the distal end side of the cannula 56 is coated with an insulating material (for example, a fluororesin). An indicator 64A (for example, a color and/or a pattern) that can specify the insulating coating region 64 is attached to the insulating coating region 64. The indicator 64A is an example of the “indicator” according to the technology of the present disclosure.

In a case in which the treatment target part 23A is incised by the incision wire 57, the other end of the incision wire 57 is pulled by the doctor 14. Therefore, tension is given to the incision wire 57, and the incision wire 57 is stretched. In a case in which the incision wire 57 (that is, a region other than the insulating coating region 64 in the conductor 60) is brought into contact with the treatment target part 23A and energization is performed on the incision wire 57 in the stretched state, the treatment target part 23A is incised by the incision wire 57.

The duodenoscope apparatus main body 18 is connected to the control device 22 and the light source device 24 through a universal cord 66. The image processing device 25, the high-frequency cautery power supply device 61, and a receiving device 68 are connected to the control device 22. In addition, the display device 13 is connected to the image processing device 25. That is, the control device 22 is connected to the display device 13 through the image processing device 25.

In addition, here, the image processing device 25 is given as an example of an external device for expanding the functions of the control device 22. Therefore, an example of a form in which the control device 22 and the display device 13 are indirectly connected to each other through the image processing device 25 is given. However, this is only an example. For example, the display device 13 may be directly connected to the control device 22. In this case, for example, the functions of the image processing device 25 may be provided in the control device 22, or the control device 22 may be provided with a function of directing a server (not illustrated) to perform the same process as the process (for example, a medical support process which will be described below) performed by the image processing device 25, receiving the processing result of the server, and using the processing result.

The receiving device 68 receives an instruction from the doctor 14 and outputs the received instruction as an electric signal to the control device 22. Examples of the receiving device 68 include a keyboard, a mouse, a touch panel, a foot switch, a microphone, and/or a remote control device.

The control device 22 controls the light source device 24, transmits and receives various signals to and from the camera 48, or transmits and receives various signals to and from the image processing device 25.

The light source device 24 emits light under the control of the control device 22 and supplies the light to the illumination device 50. A light guide is provided in the illumination device 50, and the light supplied from the light source device 24 is emitted from the illumination window 50A via the light guide. The control device 22 directs the camera 48 to perform imaging, acquires the captured image 40 (see FIG. 1) from the camera 48, and outputs the captured image 40 to a predetermined output destination (for example, the image processing device 25).

The image processing device 25 performs various types of image processing on the captured image 40 input from the control device 22. The image processing device 25 outputs the captured image 40 subjected to various types of image processing to a predetermined output destination (for example, the display device 13).

In addition, here, an example of the form in which the captured image 40 output from the control device 22 is output to the display device 13 through the image processing device 25 has been described. However, this is only an example. For example, the control device 22 and the display device 13 may be connected to each other, and the captured image 40 subjected to the image processing by the image processing device 25 may be displayed on the display device 13 through the control device 22.

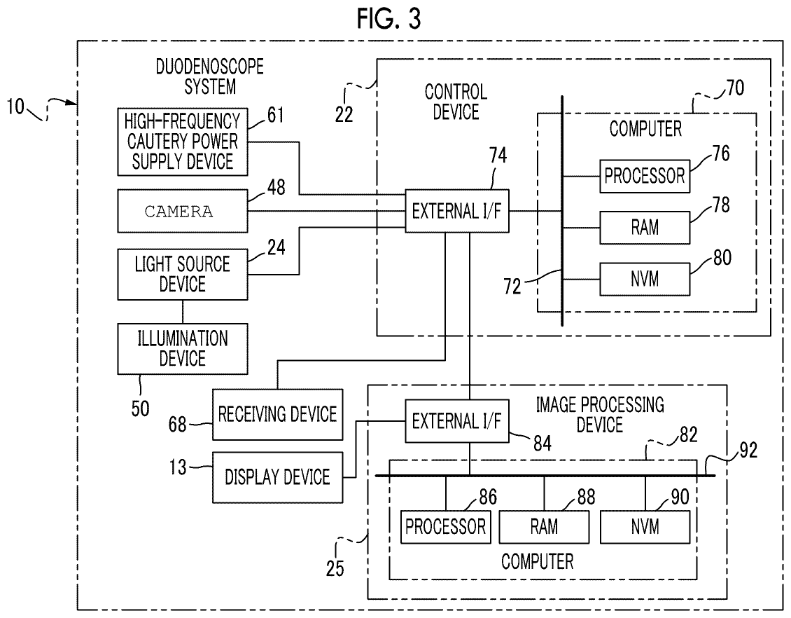

For example, as illustrated in FIG. 3, the control device 22 comprises a computer 70, a bus 72, and an external I/F 74. The computer 70 comprises a processor 76, a RAM 78, and an NVM 80. The processor 76, the RAM 78, the NVM 80, and the external I/F 74 are connected to the bus 72.

For example, the processor 76 includes a CPU and a GPU and controls the entire control device 22. The GPU operates under the control of the CPU and is in charge of, for example, performing various processes of a graphic system and performing calculation using a neural network. In addition, the processor 76 may be one or more CPUs with which the functions of the GPU have been integrated or may be one or more CPUs with which the functions of the GPU have not been integrated.

The RAM 78 is a memory that temporarily stores information and that is used as a work memory by the processor 76. The NVM 80 is a non-volatile storage device that stores, for example, various programs and various parameters. An example of the NVM 80 is a flash memory (for example, an EEPROM and/or an SSD). In addition, the flash memory is only an example and may be other non-volatile storage devices, such as HDDs, or a combination of two or more types of non-volatile storage devices.

The external I/F 74 transmits and receives various types of information between one or more devices (hereinafter, also referred to as “first external devices”) outside the control device 22 and the processor 76. An example of the external I/F 74 is a USB interface.

As one of the first external devices, the camera 48 is connected to the external I/F 74, and the external I/F 74 transmits and receives various types of information between the camera 48 and the processor 76. The processor 76 controls the camera 48 through the external I/F 74. In addition, the processor 76 acquires the captured image 40 (see FIG. 1) obtained by imaging the inside of the subject 20 with the camera 48 through the external I/F 74.

As one of the first external devices, the high-frequency cautery power supply device 61 is connected to the external I/F 74, and the external I/F 74 transmits and receives various types of information between the high-frequency cautery power supply device 61 and the processor 76. The processor 76 controls the high-frequency cautery power supply device 61 and acquires a signal indicating an operating state of the high-frequency cautery power supply device 61 from the high-frequency cautery power supply device 61.

As one of the first external devices, the light source device 24 is connected to the external I/F 74, and the external I/F 74 transmits and receives various types of information between the light source device 24 and the processor 76. The light source device 24 supplies light to the illumination device 50 under the control of the processor 76. The illumination device 50 performs irradiation with the light supplied from the light source device 24.

As one of the first external devices, the receiving device 68 is connected to the external I/F 74. The processor 76 acquires the instruction received by the receiving device 68 through the external I/F 74 and performs a process corresponding to the acquired instruction.

The image processing device 25 comprises a computer 82 and an external I/F 84. The computer 82 comprises a processor 86, a RAM 88, and an NVM 90. The processor 86, the RAM 88, the NVM 90, and the external I/F 84 are connected to a bus 92. In this embodiment, the image processing device 25 is an example of a “medical support device” according to the technology of the present disclosure, the computer 82 is an example of a “computer” according to the technology of the present disclosure, and the processor 86 is an example of a “processor” according to the technology of the present disclosure.

In addition, since a hardware configuration of the computer 82 (that is, the processor 86, the RAM 88, and the NVM 90) is basically the same as a hardware configuration of the computer 70, the description of the hardware configuration of the computer 82 will not be repeated here.

The external I/F 84 transmits and receives various types of information between one or more devices (hereinafter, also referred to as “second external devices”) outside the image processing device 25 and the processor 86. A USB interface is given as an example of the external I/F 84.

As one of the second external devices, the control device 22 is connected to the external I/F 84. In the example illustrated in FIG. 3, the external I/F 74 of the control device 22 is connected to the external I/F 84. The external I/F 84 transmits and receives various types of information between the processor 86 of the image processing device 25 and the processor 76 of the control device 22. For example, the processor 86 acquires the captured image 40 (see FIG. 1) from the processor 76 of the control device 22 through the external I/Fs 74 and 84 and performs various types of image processing on the acquired captured image 40.

As one of the second external devices, the display device 13 is connected to the external I/F 84. The processor 86 controls the display device 13 through the external I/F 84 such that various types of information (for example, the captured image 40 subjected to various types of image processing) is displayed on the display device 13.

Meanwhile, in a case in which the doctor 14 performs treatment using the papillotomy knife 54 on the treatment target part 23A, the doctor 14 operates the papillotomy knife 54 while observing the captured image 40 displayed on the screen 36. In a case in which the treatment target part 23A is incised by the incision wire 57, the incision wire 57 is energized. Here, in order to safely perform the treatment using the papillotomy knife 54 on the treatment target part 23A, it is necessary to energize the incision wire 57 in a state in which the papillotomy knife 54 protrudes from the treatment opening 52 to a safe position. The reason is that, for example, in a case in which the incision wire 57 is energized in a state in which the conductive portion of the incision wire 57 is in contact with the conductive region 53A of the insertion passage 53, sparks occur, which may cause the damage of, for example, the components of the duodenoscope apparatus 12 and/or the intestinal wall 23.

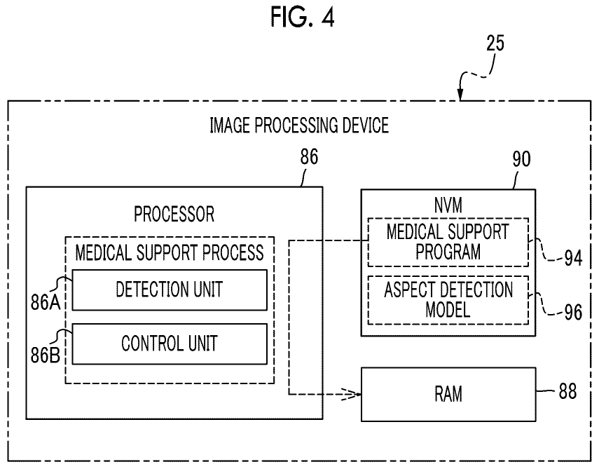

Therefore, in view of these circumstances, in this embodiment, for example, as illustrated in FIG. 4, the processor 86 of the image processing device 25 performs the medical support process.

A medical support program 94 is stored in the NVM 90. The medical support program 94 is an example of a “program” according to the technology of the present disclosure. The processor 86 reads the medical support program 94 from the NVM 90 and executes the read medical support program 94 on the RAM 88 to perform the medical support process. The processor 86 operates as a detection unit 86A and a control unit 86B in accordance with the medical support program 94 executed on the RAM 88 to implement the medical support process.

An aspect detection model 96 is stored in the NVM 90. The aspect detection model 96 is used by the detection unit 86A, which will be described in detail below.

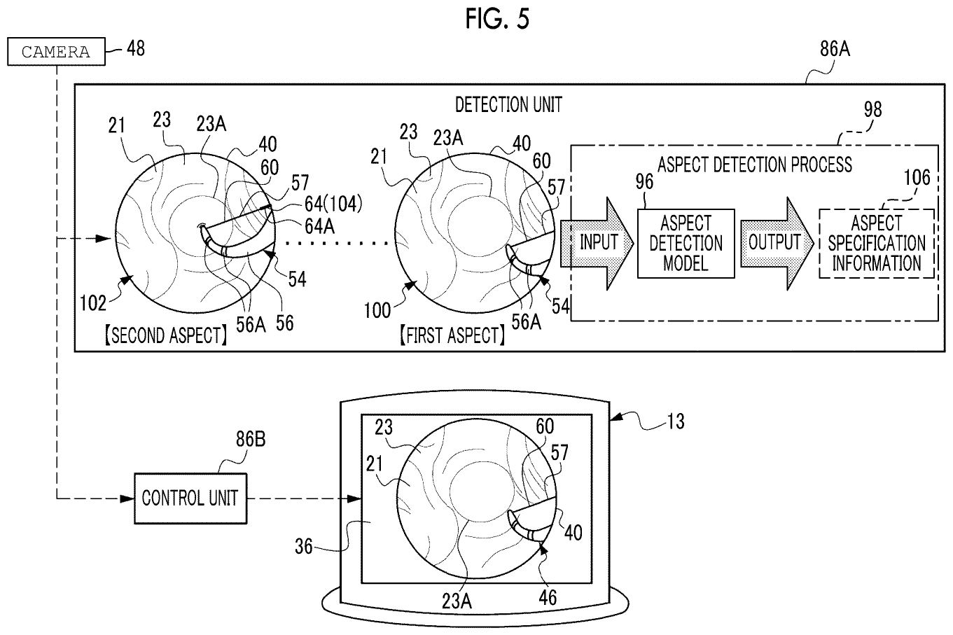

For example, as illustrated in FIG. 5, the detection unit 86A and the control unit 86B acquire the captured image 40, which has been captured by the camera 48 at an imaging frame rate (for example, several tens of frames/sec), from the camera 48 frame by frame.

The control unit 86B displays the captured image 40 as a live view image on the screen 36. That is, each time the control unit 86B acquires the captured image 40 from the camera 48 frame by frame, the control unit 86B sequentially displays the acquired captured image 40 on the screen 36 according to a display frame rate (for example, several tens of frames/sec).

The detection unit 86A performs an aspect detection process 98 on the captured image 40 acquired from the camera 48 to detect an aspect in the duodenum 21 included in the captured image 40. The aspect in the duodenum 21 is classified into a plurality of aspects including a first aspect 100 and a second aspect 102. The first aspect 100 is an aspect in which an insulating region 104 is not included in the captured image 40. The second aspect 102 is an aspect in which the treatment target part 23A, the conductor 60, and at least a portion of the insulating region 104 are included in the captured image 40.

Here, the insulating region 104 means a region that insulates the conductor 60 (that is, a region that is electrically insulated from the conductor 60). Examples of the insulating region 104 include the insulating sheath 62 (see FIG. 3) and the insulating coating region 64. The insulating region 104 is an example of an “insulating region” according to the technology of the present disclosure.

The aspect detection process 98 is an AI-type image recognition process. Here, a process using the aspect detection model 96 is performed as the aspect detection process 98.

The aspect detection model 96 is a trained model for AI-type object detection and is optimized by performing machine learning using first training data on a neural network. The first training data is a plurality of data items (that is, data corresponding to a plurality of frames) in which first example data and first correct answer data have been associated with each other.

The first example data is an image corresponding to the captured image 40. The first correct answer data is correct answer data (that is, an annotation) for the first example data. Here, an annotation that specifies, as the first aspect 100, an aspect in which the insulating region 104 is not included and that specifies, as the second aspect 102, an aspect in which the treatment target part 23A, the conductor 60, and at least a portion of the insulating region 104 are included is used as an example of the first correct answer data.

That is, the annotation used as the first correct answer data is information that distinguishably specifies the first aspect 100 and the second aspect 102 and that is used as the first correct answer data. Therefore, the aspect detection model 96 is constructed as a model that distinguishably detects the first aspect 100 and the second aspect 102 from the captured image 40.

The detection unit 86A acquires the captured image 40 from the camera 48 and inputs the acquired captured image 40 to the aspect detection model 96. Then, the aspect detection model 96 outputs aspect specification information 106 that specifies the aspect in the duodenum 21 included in the input captured image 40. That is, the aspect specification information 106 output from the aspect detection model 96 is information that distinguishably specifies the first aspect 100 and the second aspect 102. The detection unit 86A acquires the aspect specification information 106 output from the aspect detection model 96 and detects the aspect in the duodenum 21 included in the captured image 40. That is, the detection unit 86A detects the first aspect 100 or the second aspect 102 as the aspect in the duodenum 21 included in the captured image 40.

In addition, here, an example of the form in which the aspect in which the treatment target part 23A, the conductor 60, and at least a portion of the insulating region 104 are included in the captured image 40 is set as the second aspect 102 and the first correct answer data is created according to this aspect has been described. However, this is only an example. For example, an aspect in which the treatment target part 23A, the conductor 60, one indicator 56A or a plurality of indicators 56A, and at least a portion of the insulating region 104 are included in the captured image 40 may be set as the second aspect 102, and the first correct answer data may be created according to this aspect. In addition, for example, an aspect in which the papillotomy knife 54 that is in contact with the treatment target part 23A, the conductor 60, one indicator 56A or a plurality of indicators 56A, and at least a portion of the insulating region 104 are included in the captured image 40 may be set as the second aspect 102, and the first correct answer data may be created according to this aspect.

Further, here, an example of the form in which the AI-type image recognition process is performed as the aspect detection process 98 has been described. However, this is only an example. A non-AI-type image recognition process (for example, template matching) may be performed as the aspect detection process 98 to detect the aspect in the duodenum 21 included in the captured image 40.

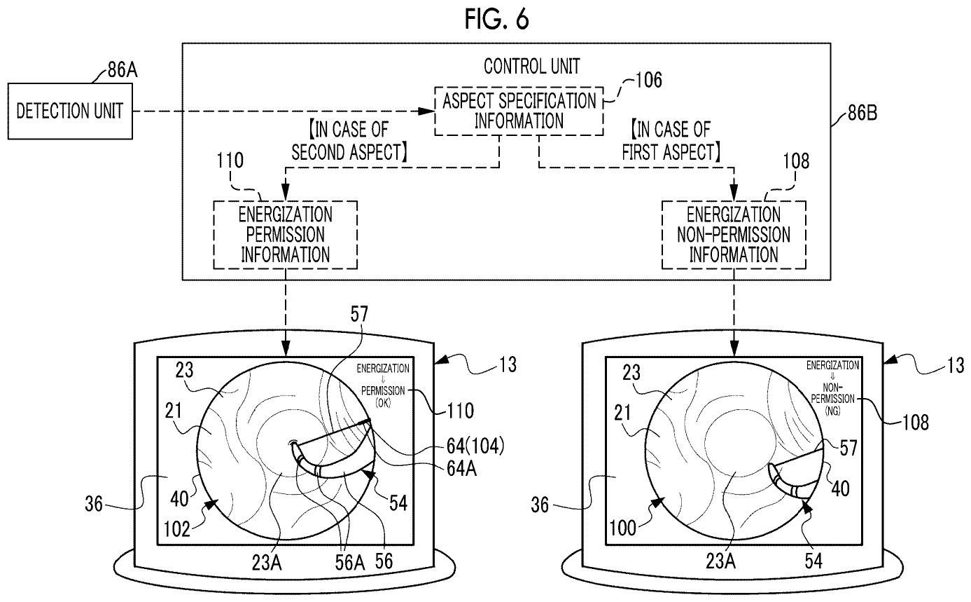

For example, as illustrated in FIG. 6, the control unit 86B displays the captured image 40 on the screen 36 and selectively displays energization non-permission information 108 and energization permission information 110 on the screen 36 on the basis of the aspect specification information 106 acquired by the detection unit 86A using the captured image 40 displayed on the screen 36. In order to achieve this, for example, the control unit 86B determines whether or not to permit energization according to the aspect in which the papillotomy knife 54 is included in the captured image 40. That is, the control unit 86B determines whether or not to permit energization on the basis of the aspect specification information 106. Then, the control unit 86B selects the energization non-permission information 108 or the energization permission information 110 on the basis of the determination result and displays the selected information on the screen 36. Here, the energization non-permission information 108 and the energization permission information 110 are examples of “permission and non-permission information” according to the technology of the present disclosure.

The control unit 86B displays the energization non-permission information 108 on the screen 36 in a case in which the first aspect 100 is specified by the aspect specification information 106 acquired by the detection unit 86A. In the example illustrated in FIG. 6, the energization non-permission information 108 is displayed at a position adjacent to the captured image 40. The energization non-permission information 108 is information indicating that the energization of the incision wire 57 is not permitted. In the example illustrated in FIG. 6, text information notifying that the energization of the incision wire 57 is not permitted is illustrated as an example of the energization non-permission information 108 displayed on the screen 36. However, this is only an example, and the energization non-permission information 108 may be an image (for example, an image including a color and/or a pattern) notifying that the energization of the incision wire 57 is not permitted.

The control unit 86B displays the energization permission information 110 on the screen 36 in a case in which the second aspect 102 is specified by the aspect specification information 106 acquired by the detection unit 86A. In the example illustrated in FIG. 6, the energization permission information 110 is displayed at a position adjacent to the captured image 40. The energization permission information 110 is information indicating that the energization of the incision wire 57 is permitted. In the example illustrated in FIG. 6, text information notifying that the energization of the incision wire 57 is permitted is illustrated as an example of the energization permission information 110 displayed on the screen 36. However, this is only an example, and the energization permission information 110 may be an image (for example, an image including a color and/or a pattern) notifying that the energization of the incision wire 57 is permitted.

In the example illustrated in FIG. 6, the energization non-permission information 108 or the energization permission information 110 is displayed at a position adjacent to the captured image 40. However, this is only an example, and the energization non-permission information 108 or the energization permission information 110 may be displayed at other positions.

Further, in the example illustrated in FIG. 6, the display device 13 is illustrated as the output destination of the energization non-permission information 108 or the energization permission information 110, and the energization non-permission information 108 or the energization permission information 110 is displayed on the screen 36. However, this is only an example, and the output destination of the energization non-permission information 108 or the energization permission information 110 may be devices other than the display device 13. Examples of the output destination of the energization non-permission information 108 or the energization permission information 110 include a sound reproducing device, a printing device, a server, and a terminal device. For example, the energization non-permission information 108 or the energization permission information 110 may be output as sound by the sound reproducing device or may be printed as text or the like on a recording medium (for example, paper) or the like by the printing device. In addition, the energization non-permission information 108 or the energization permission information 110 may be stored in the server or the terminal device or may be stored in an electronic medical record, together with the captured image 40 displayed on the screen 36.

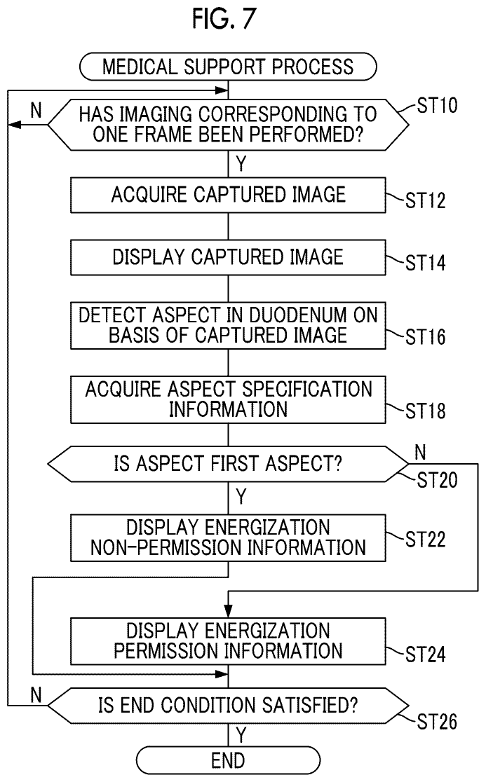

Next, the operation of a portion of the duodenoscope system 10 according to the technology of the present disclosure will be described with reference to FIG. 7.

FIG. 7 illustrates an example of a flow of the medical support process performed by the processor 86. The flow of the medical support process illustrated in FIG. 7 is an example of a “medical support method” according to the technology of the present disclosure.

In the example illustrated in FIG. 7, in Step ST10, the detection unit 86A determines whether or not imaging corresponding to one frame has been performed on the duodenum 21 by the camera 48. In a case in which the imaging corresponding to one frame has not been performed on the duodenum 21 by the camera 48 in Step ST10, the determination result is “No”, and the determination in Step ST10 is performed again. In a case in which the imaging corresponding to one frame has been performed on the duodenum 21 by the camera 48 in Step ST10, the determination result is “Yes”, and the medical support process proceeds to Step ST12.

In Step ST12, the detection unit 86A and the control unit 86B acquire the captured image 40 corresponding to one frame obtained by imaging the duodenum 21 with the camera 48 (see FIG. 5). After the process in Step ST12 is performed, the medical support process proceeds to Step ST14.

In Step ST14, the control unit 86B displays the captured image 40 acquired in Step ST12 on the screen 36 (see FIGS. 5 and 6). After the process in Step ST14 is performed, the medical support process proceeds to Step ST16.

In Step ST16, the detection unit 86A performs the aspect detection process 98 using the captured image 40 acquired in Step ST12 to detect the aspect in the duodenum 21 included in the captured image 40 (see FIG. 5). After the process in Step ST16 is performed, the medical support process proceeds to Step ST18.

In Step ST18, the detection unit 86A acquires the aspect specification information 106 that specifies the aspect in the duodenum 21 included in the captured image 40 (see FIG. 6). After the process in Step ST18 is performed, the medical support process proceeds to Step ST20.

In Step ST20, the control unit 86B determines whether or not the aspect in the duodenum 21 included in the captured image 40 acquired in Step ST12 is the first aspect 100 on the basis of the aspect specification information 106 acquired in Step ST18. In a case in which the aspect in the duodenum 21 included in the captured image 40 acquired in Step ST12 is not the first aspect 100 in Step ST20 (for example, in a case in which the aspect is the second aspect 102), the determination result is “No”, and the medical support process proceeds to Step ST24. In a case in which the aspect in the duodenum 21 included in the captured image 40 acquired in Step ST12 is the first aspect 100 in Step ST20, the determination result is “Yes”, and the medical support process proceeds to Step ST22.

In Step ST22, the control unit 86B displays the energization non-permission information 108 on the screen 36 (see FIG. 6). After the process in Step ST22 is performed, the medical support process proceeds to Step ST26.

In Step ST24, the control unit 86B displays the energization permission information 110 on the screen 36 (see FIG. 6). After the process in Step ST24 is performed, the medical support process proceeds to Step ST26.

In Step ST26, the control unit 86B determines whether or not a medical support process end condition is satisfied. An example of the medical support process end condition is a condition that an instruction for the duodenoscope system 10 to end the medical support process is given (for example, a condition that the receiving device 68 receives an instruction to end the medical support process).

In a case in which the medical support process end condition is not satisfied in Step ST26, the determination result is “No”, and the medical support process proceeds to Step ST10. In a case in which the medical support process end condition is satisfied in Step ST26, the determination result is “Yes”, and the medical support process ends.

As described above, in the duodenoscope system 10, the energization non-permission information 108 and the energization permission information 110 are selectively output as the information indicating whether or not to permit the energization of the incision wire 57 on the basis of the captured image 40 obtained by imaging, with the camera 48, the inside of the duodenum 21 including the papillotomy knife 54. This enables the doctor 14 to energize the incision wire 57 in a safe state with reference to the energization non-permission information 108 or the energization permission information 110.

In addition, in the duodenoscope system 10, it is determined whether or not to permit the energization of the incision wire 57 on the basis of an image including the conductor 60 and the insulating region 104 as the captured image 40, and the energization non-permission information 108 and the energization permission information 110 are selectively output on the basis of the determination result. Therefore, it is possible to perform energization without a concern caused by performing the energization in a state in which, of the conductor 60 and the insulating region 104, only the conductor 60 is included in the captured image 40 (for example, a concern that sparks will occur due to energization in a state in which the conductor 60 does not protrude from the treatment opening 52).

In addition, in the duodenoscope system 10, it is determined whether or not to permit the energization of the incision wire 57 on the basis of an image including one or more indicators 56A, the indicator 62A, and/or the indicator 64A as the captured image 40, and the energization non-permission information 108 and the energization permission information 110 are selectively output on the basis of the determination result. Therefore, it is possible to perform energization without a concern caused by performing the energization in a state in which none of one or more indicators 56A, the indicator 62A, and the indicator 64A are included in the captured image 40 (for example, a concern that sparks will occur due to energization in a state in which the conductor 60 does not protrude from the treatment opening 52).

Further, in the duodenoscope system 10, the treatment target part 23A is the duodenal papilla. Therefore, in a case in which the duodenal papilla is treated (for example, incised) by the papillotomy knife 54, the doctor 14 can energize the incision wire 57 in a safe state with reference to the energization non-permission information 108 or the energization permission information 110.

Further, in the duodenoscope system 10, the energization non-permission information 108 and the energization permission information 110 are selectively displayed on the screen 36. Therefore, the doctor 14 can visually ascertain whether or not energization is permitted.

Furthermore, in the duodenoscope system 10, the energization non-permission information 108 and the energization permission information 110 are selectively displayed on the screen 36, and the captured image 40 is displayed on the screen 36. Therefore, the doctor 14 energizes the incision wire 57 in a safe state while visually ascertaining the position of the papillotomy knife 54 in the duodenum 21 through the captured image 40 displayed on the screen 36.

In addition, in the duodenoscope system 10, it is detected whether the aspect in the duodenum 21 is the first aspect 100 or the second aspect 102 on the basis of the captured image 40 including the conductor 60. Therefore, it is possible to detect whether the aspect in the duodenum 21 is the first aspect 100 or the second aspect 102 with high accuracy, as compared to a case in which it is detected whether the aspect in the duodenum 21 is the first aspect 100 or the second aspect 102 on the basis of the captured image 40 in which the conductor 60 is not included.

Further, in the duodenoscope system 10, the energization of the incision wire 57 is permitted in a case in which the conductor 60 and the insulating region 104 are included in the captured image 40. Therefore, it is possible to prevent the incision wire 57 from being energized in a state in which the conductor 60 is located in the insertion passage 53. As a result, it is possible to prevent sparks caused by the energization of the incision wire 57 in a state in which the conductor 60 and the conductive region 53A in the insertion passage 53 are in contact with each other.

Further, in the duodenoscope system 10, the indicators 62A and 64A are given to the insulating region 104. Therefore, it is possible to achieve the high-accuracy aspect detection process 98 as compared to a case in which the indicators 62A and 64A are not given to the insulating region 104. In addition, the doctor 14 can easily ascertain the position of the insulating region 104 through the captured image 40 displayed on the screen 36.

Further, in the duodenoscope system 10, the insertion passage 53 has the conductive region 53A, and the papillotomy knife 54 protrudes from the treatment opening 52 through the insertion passage 53. Sparks occur in a case in which the incision wire 57 is energized in a state in which the conductor 60 of the incision wire 57 of the papillotomy knife 54 is in contact with the conductive region 53A of the insertion passage 53. On the other hand, in a case in which the papillotomy knife 54 protrudes from the treatment opening 52 such that the insulating region 104 is exposed, the insulating region 104 is in contact with the conductive region 53A. Therefore, even in a case in which the incision wire 57 is energized, no sparks occur. Therefore, in the duodenoscope system 10, it is determined whether or not to permit the energization of the incision wire 57 according to the aspect in which the papillotomy knife 54 is included in the captured image 40 (see FIG. 6). Therefore, it is possible to prevent a situation in which sparks occur due to the energization of the incision wire 57 in a state in which the conductor 60 and the conductive region 53A in the insertion passage 53 are in contact with each other.

Further, in the above-described embodiment, an example of the form in which the aspect detection process 98 is performed even in a case in which the doctor 14 does not give an instruction to the duodenoscope system 10 and the energization non-permission information 108 or the energization permission information 110 is output to the display device 13 even in a case in which the doctor 14 does not give an instruction to the duodenoscope system 10 has been described. However, the technology of the present disclosure is not limited thereto. For example, the aspect detection process 98 may be performed in a case in which the doctor 14 gives an instruction to the duodenoscope system 10, and the energization non-permission information 108 or the energization permission information 110 may be output to the display device 13 in a case in which the doctor 14 gives an instruction to the duodenoscope system 10.

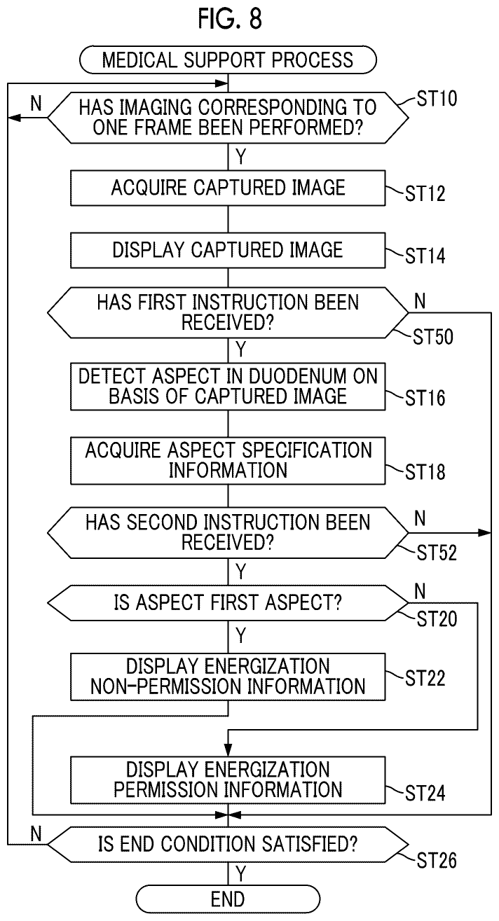

In this case, for example, a medical support process illustrated in FIG. 8 is performed by the processor 86. The medical support process illustrated in FIG. 8 differs from the medical support process illustrated in FIG. 7 in that it has a process in Step ST50 between the process in Step ST14 and the process in Step ST16 and has a process in Step ST52 between the process in Step ST18 and the process in Step ST20.

In the medical support process illustrated in FIG. 8, in Step ST50, the detection unit 86A determines whether or not a first instruction has been received by the receiving device 68. The first instruction means, for example, an instruction to direct the detection unit 86A to perform the aspect detection process 98. The first instruction received by the receiving device 68 is an example of a “first instruction” according to the technology of the present disclosure.

In a case in which the first instruction has not been received by the receiving device 68 in Step ST50, the determination result is “No”, and the medical support process proceeds to Step ST26. In a case in which the first instruction has been received by the receiving device 68 in Step ST50, the determination result is “Yes”, and the medical support process proceeds to Step ST16.

In the medical support process illustrated in FIG. 8, in Step ST52, the control unit 86B determines whether or not a second instruction has been received by the receiving device 68. The second instruction means, for example, an instruction to output the energization non-permission information 108 or the energization permission information 110 to a predetermined output destination (for example, the display device 13). The second instruction received by the receiving device 68 is an example of a “second instruction” according to the technology of the present disclosure.

In a case in which the second instruction has not been received by the receiving device 68 in Step ST52, the determination result is “No”, and the medical support process proceeds to Step ST26. In a case in which the second instruction has been received by the receiving device 68 in Step ST52, the determination result is “Yes”, and the medical support process proceeds to Step ST20.

In the medical support process illustrated in FIG. 8, the aspect detection process 98 is performed in Step ST16 by performing the process in Step ST50. In addition, by performing the process in Step ST52, the energization non-permission information 108 or the energization permission information 110 is output to the display device 13, and the energization non-permission information 108 and the energization permission information 110 are selectively displayed on the display device 13. Therefore, it is possible to perform the aspect detection process 98 at a time intended by the doctor 14, and it is possible to selectively display the energization non-permission information 108 and the energization permission information 110 on the display device 13 at a time intended by the doctor 14. As a result, it is possible to suppress energy consumption associated with performing the aspect detection process 98, and it is possible to reduce a feeling of discomfort that is visually given to the doctor 14 due to the energization non-permission information 108 or the energization permission information 110 always being displayed on the screen 36.

In the above-described embodiment, an example of the form in which the aspect in the duodenum 21 is detected on the basis of the presence or absence of the aspect in which the treatment target part 23A, the conductor 60, and at least a portion of the insulating region 104 are included in the captured image 40 and it is determined whether or not to permit energization on the basis of the detection result has been described. However, the technology of the present disclosure is not limited thereto. For example, it may be determined whether or not to permit energization on the basis of the size and position of the papillotomy knife 54 included in the captured image 40.

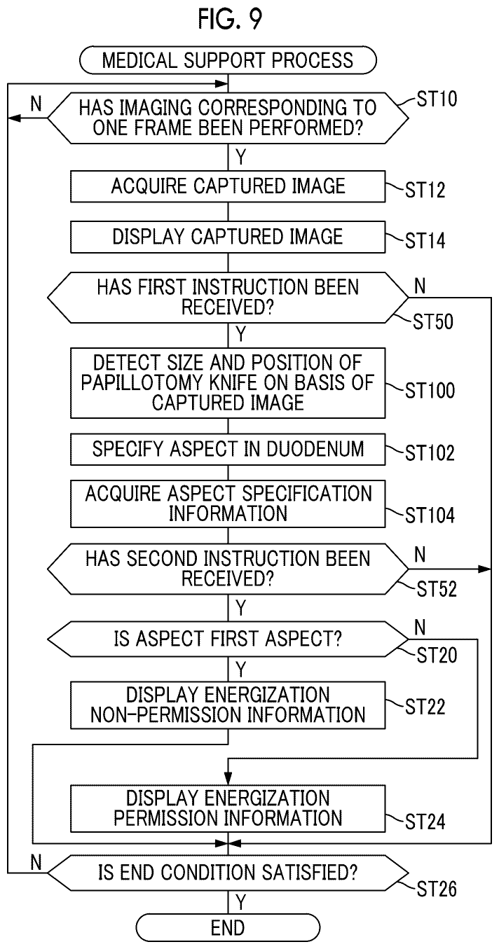

In this case, for example, a medical support process illustrated in FIG. 9 is performed by the processor 86. The medical support process illustrated in FIG. 9 differs from the medical support process illustrated in FIG. 8 in that it has processes in Steps ST100 to ST104 instead of the process in Step ST16 and the process in Step ST18.

In the medical support process illustrated in FIG. 9, in Step ST100, the detection unit 86A detects the size of the papillotomy knife 54 (for example, the length of the incision wire 57) in the captured image 40 and the position of the papillotomy knife 54 (for example, the position of the incision wire 57) in the captured image 40 on the basis of the captured image 40 acquired in Step ST12. The detection of the size and position of the papillotomy knife 54 is achieved, for example, by performing a process including the AI-type image recognition process or the non-AI-type image recognition process.

In Step ST102, the detection unit 86A specifies the aspect in the duodenum 21 on the basis of the detection result in Step ST100, that is, the size of the papillotomy knife 54 in the captured image 40 and the position of the papillotomy knife 54 in the captured image 40. For example, in a case in which the size of the papillotomy knife 54 in the captured image 40 and the position of the papillotomy knife 54 in the captured image 40 are the size and position where the treatment target part 23A, the conductor 60, and at least a portion of the insulating region 104 are not included in the captured image 40, the detection unit 86A specifies the aspect in the duodenum 21 as the first aspect 100. In addition, in a case in which the size of the papillotomy knife 54 in the captured image 40 and the position of the papillotomy knife 54 in the captured image 40 are the size and position where the treatment target part 23A, the conductor 60, and at least a portion of the insulating region 104 are included, the detection unit 86A specifies the aspect in the duodenum 21 as the second aspect 102.

In addition, the size of the papillotomy knife 54 in the captured image 40 may be defined by, for example, the length of the conductor 60 of the incision wire 57 (that is, the length of a portion of the conductor 60 on the distal end side with respect to the insulating coating region 64) or may be defined by the ratio of the length of the conductor 60 included in the captured image 40 to the total length of the conductor 60 of the incision wire 57 (for example, the length of a portion on the distal end side with respect to the insulating sheath 62). Further, the position of the papillotomy knife 54 in the captured image 40 may be defined by, for example, the position of the indicator 56A and/or the indicator 62A in the captured image 40 or may be defined by the position of a specific portion (for example, a distal end of the cannula 56 or a joint point between the cannula 56 and the incision wire 57) other than the indicator 56A and/or the indicator 62A in the captured image 40.

In Step ST104, the detection unit 86A acquires the aspect specification information 106. The acquisition of the aspect specification information 106 is achieved by the generation of information indicating the specification result in Step ST102 (that is, information specifying whether the aspect in the duodenum 21 is the first aspect 100 or the second aspect 102) as the aspect specification information 106.

As described above, even in a case in which it is determined whether or not to permit energization on the basis of the size and position of the papillotomy knife 54 included in the captured image 40, the same effects as those in the above-described embodiment can be obtained.

Further, here, an example of the form in which it is determined whether or not to permit energization on the basis of the size and position of the papillotomy knife 54 included in the captured image 40 has been described. However, this is only an example, and it may be determined whether or not to permit energization on the basis of the size or position of the papillotomy knife 54 included in the captured image 40.

In addition, the size of the papillotomy knife 54 may be defined by the length of the conductor 60 (that is, a portion of the conductor 60 on the distal end side with respect to the insulating coating region 64), may be defined by the length of the insulating region 104, or may be defined by the length of the insulating coating region 64. In this case, the same effects as those in the above-described embodiment can be obtained.

Further, the position of the papillotomy knife 54 may be defined by the position of the conductor 60 (that is, a portion of the conductor 60 on the distal end side with respect to the insulating coating region 64), may be defined by the position of the insulating region 104, or may be defined by the position of the insulating coating region 64. In this case, the same effects as those in the above-described embodiment can be obtained.

In the above-described embodiment, an example of the form in which it is assumed that the doctor 14 gives an instruction to energize the incision wire 57 (hereinafter, referred to as an “energization instruction”) with reference to the information related to whether or not to permit the energization which is displayed on the screen 36 has been described. However, even in a case in which the energization instruction is given to the duodenoscope system 10, the energization may or may not be performed depending on the state of the incision wire 57.

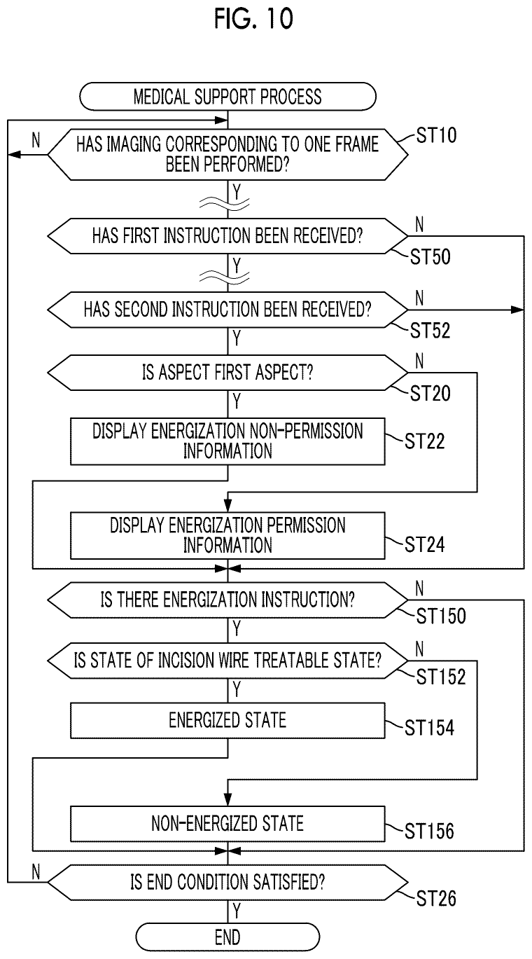

In this case, for example, the medical support process illustrated in FIG. 10 is performed by the processor 86. The medical support process illustrated in FIG. 10 differs from the medical support process illustrated in FIG. 9 in that it has processes in Steps ST150 to ST156 between the process in Step ST24 and the process in Step ST26.

In the medical support process illustrated in FIG. 10, in Step ST150, the control unit 86B determines whether or not the energization instruction has been received by the receiving device 68. In a case in which the energization instruction has not been received by the receiving device 68 in Step ST150, the determination result is “No”, and the medical support process proceeds to Step ST26. In a case in which the energization instruction has been received by the receiving device 68 in Step ST150, the determination result is “Yes”, and the medical support process proceeds to Step ST152.

In Step ST152, the control unit 86B determines whether or not the state of the incision wire 57 included in the captured image 40 acquired in Step ST12 is a treatable state in which the treatment target part 23A can be treated (for example, incised) by energization. The treatable state means, for example, a state in which the incision wire 57 is stretched. In addition, the determination of whether or not the state is the treatable state is achieved by performing a process including the AI-type image recognition process or the non-AI-type image recognition process (for example, a process including an image recognition process that distinguishably detects a state in which the incision wire 57 is stretched and a state in which the incision wire 57 is loosened) on the captured image 40.

In a case in which the state of the incision wire 57 included in the captured image 40 acquired in Step ST12 is not the treatable state in Step ST152, the determination result is “No”, and the medical support process proceeds to Step ST156. In a case in which the state of the incision wire 57 included in the captured image 40 acquired in Step ST12 is the treatable state in Step ST152, the determination result is “Yes”, and the medical support process proceeds to Step ST154.

In Step ST154, the control unit 86B changes the high-frequency cautery power supply device 61 to an energized state in which the incision wire 57 is energized. After the process in Step ST154 is performed, the medical support process proceeds to Step ST26.

In Step ST156, the control unit 86B changes the high-frequency cautery power supply device 61 to a non-energized state in which the incision wire 57 is not energized. After the process in Step ST156 is performed, the medical support process proceeds to Step ST26.

As described above, by the execution of the processes in Steps ST150 to ST156, the high-frequency cautery power supply device 61 is changed to the energized state on condition that the state of the incision wire 57 is the treatable state and is changed to the non-energized state on condition that the state of the incision wire 57 is not the treatable state. Therefore, it is possible to allow the execution of treatment (for example, incision) on the treatment target part 23A in a case in which the state of the incision wire 57 is the treatable state and to prevent the execution of the treatment on the treatment target part 23A in a case in which the state of the incision wire 57 is not the treatable state.

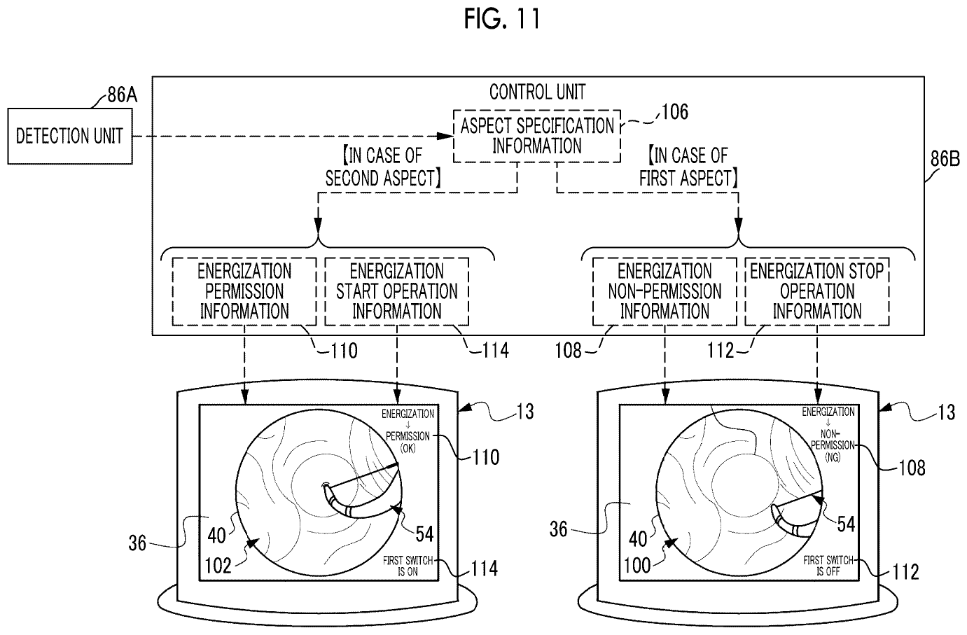

In the above-described embodiment, an example of the form in which the energization non-permission information 108 and the energization permission information 110 are selectively displayed on the screen 36 has been described. However, the technology of the present disclosure is not limited thereto. For example, as illustrated in FIG. 11, in a case in which the aspect in the duodenum 21 is the first aspect 100, the control unit 86B may display energization stop operation information 112 on the screen 36. In a case in which the aspect in the duodenum 21 is the second aspect 102, the control unit 86B may display energization start operation information 114 on the screen 36. The energization stop operation information 112 is information related to an operation required to stop energization. The energization start operation information 114 is information related to an operation required to perform energization. The operation required to perform energization means an operation of turning on a first switch (for example, a foot switch). The operation required to stop energization means an operation of turning off the first switch.

In the example illustrated in FIG. 11, the display device 13 is illustrated as the output destination of the energization stop operation information 112 and the energization start operation information 114. However, this is only an example, and the output destination of the energization stop operation information 112 and the energization start operation information 114 may be output destinations other than the display device 13, similarly to the energization non-permission information 108 and the energization permission information 110.

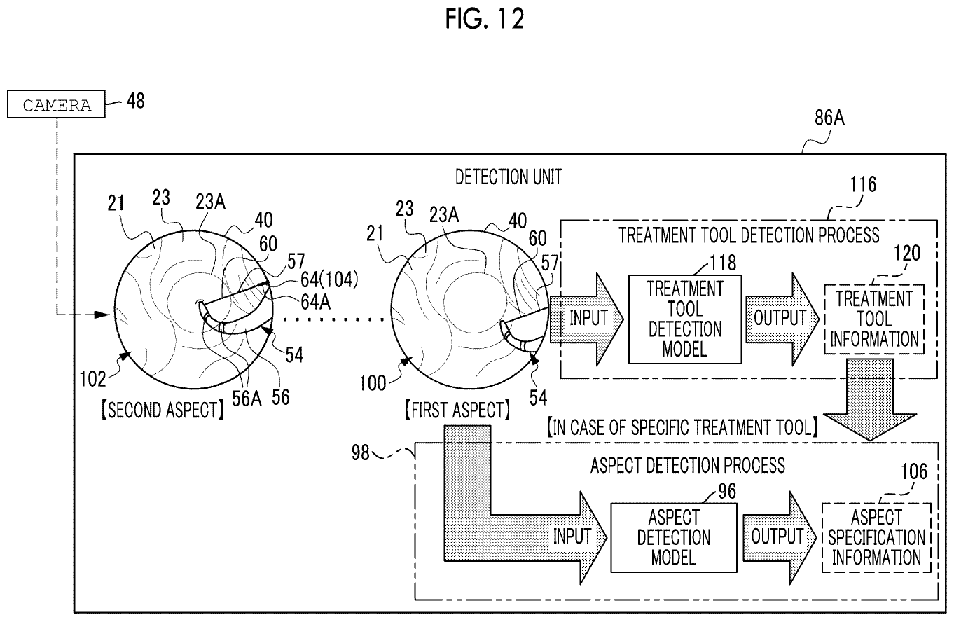

In the above-described embodiment, an example of the form in which the detection unit 86A performs the aspect detection process 98 has been described. However, for example, as illustrated in FIG. 12, the aspect detection process 98 may be performed on the basis of the result of a treatment tool detection process 116 by the detection unit 86A.

The treatment tool detection process 116 is a process of detecting the treatment tool included in the captured image 40 and is achieved by the AI-type image recognition process (that is, an object detection process) or the non-AI-type image recognition process on the captured image 40.

In the example illustrated in FIG. 12, an image recognition process using a treatment tool detection model 118 is illustrated as the treatment tool detection process 116.

The treatment tool detection model 118 is a trained model for AI-type object detection and is optimized by performing machine learning using second training data on the neural network. The second training data is a plurality of data items (that is, data corresponding to a plurality of frames) in which second example data and second correct answer data have been associated with each other. The second example data is an image corresponding to the captured image 40. The second correct answer data is correct answer data (that is, an annotation) for the second example data. Here, an annotation that specifies the type of the treatment tool included in the image used as the second example data is used as an example of the second correct answer data.

The detection unit 86A acquires the captured image 40 from the camera 48 and inputs the acquired captured image 40 to the treatment tool detection model 118. Then, the treatment tool detection model 118 outputs treatment tool information 120 that specifies the type of the treatment tool included in the input captured image 40.

The detection unit 86A acquires the treatment tool information 120 output from the treatment tool detection model 118 and specifies the type of the treatment tool included in the captured image 40 from the acquired treatment tool information 120. In a case in which the type of the treatment tool included in the captured image 40 is a specific type (for example, the papillotomy knife 54), the detection unit 86A performs the aspect detection process 98 in the same manner as that in the above-described embodiment.

As described above, the aspect detection process 98 is performed on condition that the treatment tool detection process 116 is performed to detect a specific type of treatment tool. Therefore, the aspect detection process 98 can be performed only as needed.

In addition, here, an example of the form in which the aspect detection process 98 is performed on condition that a specific type of treatment tool is detected has been described. However, this is only an example. For example, the aspect detection process 98 may be performed on condition that a specific part (for example, a pylorus or the duodenal papilla) is detected. Further, the aspect detection process 98 may be performed on condition that a portion of the conductor 60 is detected. Furthermore, the aspect detection process 98 may be performed on condition that a predetermined number of frames (for example, several frames to several tens of frames) of a specific type of treatment tool, a specific part (for example, the pylorus or the duodenal papilla), the conductor 60, or the like are detected.

In the example illustrated in FIG. 12, an example of the form in which the aspect detection process 98 is performed in a case in which the type of the treatment tool included in the captured image 40 is a specific type has been described. However, the aspect detection process 98 may be ended in a case in which the treatment on the treatment target part 23A with the treatment tool has been completed.

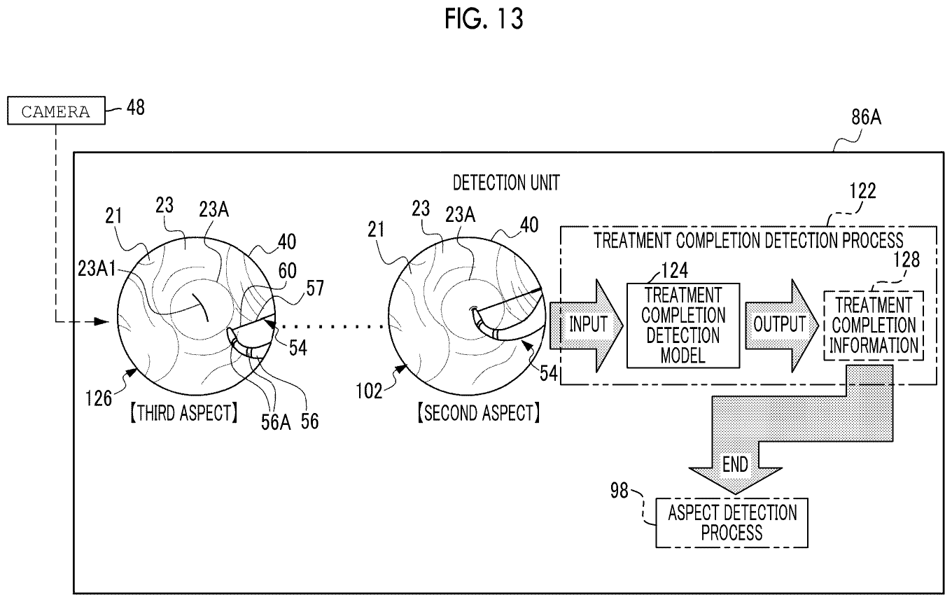

In this case, for example, as illustrated in FIG. 13, the detection unit 86A performs a treatment completion detection process 122. The treatment completion detection process 122 is, for example, a process of detecting the completion of the incision of the treatment target part 23A by the papillotomy knife 54 and is achieved by the AI-type image recognition process (that is, the object detection process) or the non-AI-type image recognition process on the captured image 40.

In the example illustrated in FIG. 13, an image recognition process using a treatment completion detection model 124 is illustrated as the treatment completion detection process 122.

The treatment completion detection model 124 is a trained model for AI-type object detection and is optimized by performing machine learning using third training data on the neural network. The third training data is a plurality of data items (that is, data corresponding to a plurality of frames) in which third example data and third correct answer data have been associated with each other. The third example data is an image corresponding to the captured image 40. The third correct answer data is correct answer data (that is, an annotation) for the third example data. Here, an annotation that specifies a third aspect 126 is used as an example of the third correct answer data. The third aspect 126 means an aspect in which an incision region 23A1 in which the treatment target part 23A has been incised by the incision wire 57 and the papillotomy knife 54 that is distant from the treatment target part 23A are included in the image used as the third example data.

The detection unit 86A acquires the captured image 40 from the camera 48 and inputs the acquired captured image 40 to the treatment completion detection model 124. Then, the treatment completion detection model 124 outputs treatment completion information 128 in a case in which the aspect included in the input captured image 40 is the third aspect 126. The treatment completion information 128 is information that specifies the third aspect 126.

In a case in which the detection unit 86A detects the third aspect 126 (that is, acquires the treatment completion information 128 output from the treatment completion detection model 124), the detection unit 86A ends the aspect detection process 98. As described above, the combined use of the treatment tool detection process 116 (see FIG. 12) and the treatment completion detection process 122 makes it possible to perform the aspect detection process 98 only as needed. As a result, it is possible to suppress energy consumption required to perform the aspect detection process 98.

In addition, during the incision of the treatment target part 23A by the incision wire 57, the control unit 86B may perform the AI-type image recognition process or the non-AI-type image recognition process to specify a direction of the incision region 23A1 (that is, an incision direction) and may switch the high-frequency cautery power supply device 61 from the energized state to the non-energized state in a case in which the direction of the incision region 23A1 is not a specific direction (for example, a direction along a traveling direction of a bile duct).

In the above-described embodiment, an example of the form in which it is determined whether or not to permit energization on the basis of whether or not the insulating region 104 is included in the captured image 40 has been described. However, the technology of the present disclosure is not limited thereto. For example, it may be determined whether or not to permit energization on the basis of the positions of one or more indicators 56A, the indicator 62A, and/or the indicator 64A and the amount of protrusion of the conductor 60 (that is, the amount of protrusion of the conductor 60 from the treatment opening 52), regardless of whether or not the insulating region 104 is included in the captured image 40. This is because there is a concern that energization will be performed in a state in which the amount of protrusion of the treatment tool (here, for example, the papillotomy knife 54) from the treatment opening 52 is less than a predetermined value (for example, a concern that sparks will occur due to energization in a state in which the amount of protrusion of the conductor 60 from the treatment opening 52 is less than a predetermined value).

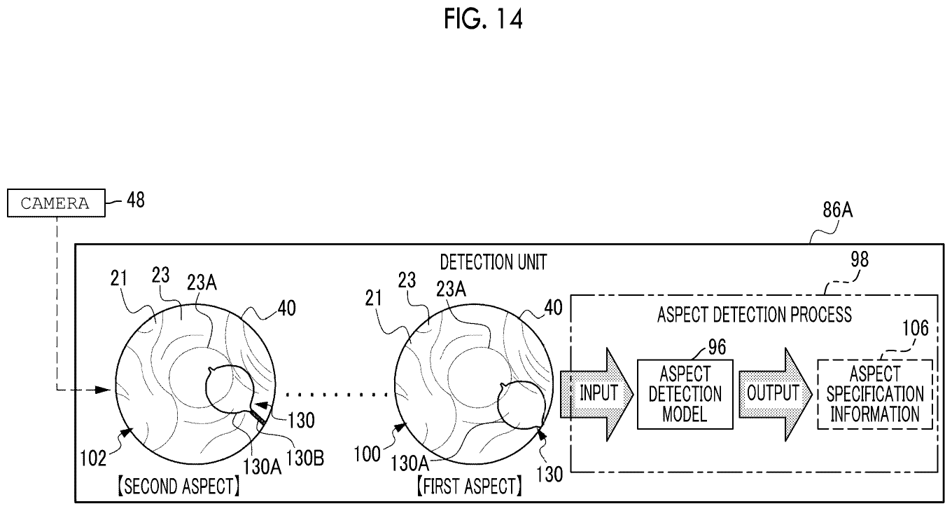

In the above-described embodiment, the papillotomy knife 54 is given as an example of the treatment tool. However, for example, even in a case in which a snare 130 is applied instead of the papillotomy knife 54 as illustrated in FIG. 14, the technology of the present disclosure is established.

The snare 130 comprises an excision wire 130A and an insulating sheath 130B. The excision wire 130A is a conductive wire and is inserted into the insulating sheath 130B. The excision wire 130A is spread in an annular shape during use. The excision wire 130A spread in the annular shape covers the periphery of a lesion and then narrows down the lesion. In this state, the excision wire 130A is energized to excise the lesion.

The excision wire 130A corresponds to the incision wire 57 of the papillotomy knife 54, and the insulating sheath 130B corresponds to the insulating sheath 62 of the papillotomy knife 54. In the example illustrated in FIG. 14, the first aspect 100 is an aspect in which the insulating sheath 130B is not included in the captured image 40. The second aspect 102 is an aspect in which the treatment target part 23A, the excision wire 130A, and at least a portion of the insulating sheath 130B are included in the captured image 40. In addition, the aspect detection model 96 may be, for example, any trained model for object detection obtained by performing machine learning, which corresponds to not only the papillotomy knife 54 but also the snare 130, or machine learning, which is specialized for the snare 130, on the neural network to optimize the neural network.

Here, the papillotomy knife 54 and the snare 130 are given as examples. However, the technology of the present disclosure is not limited thereto, but can also be applied to other treatment tools that are energized for incision or excision.

In the above-described embodiment, the duodenoscope apparatus 12 applied to the duodenum 21 is given as an example. However, the technology of the present disclosure is not limited thereto, but can also be applied to an endoscope apparatus that is used for a luminal organ, such as an esophagus, a stomach, a large intestine, or a bronchus.

In the above-described embodiment, an example of the form in which the medical support process is performed by the processor 76 of the computer 82 included in the duodenoscope apparatus 12 has been described. However, the technology of the present disclosure is not limited thereto. The device that performs the medical support process may be provided outside the duodenoscope apparatus 12. An example of the device provided outside the duodenoscope apparatus 12 is at least one server and/or at least one personal computer that is connected to the duodenoscope apparatus 12 such that it can communicate therewith. In addition, the medical support process may be dispersively performed by a plurality of devices.

Further, in the above-described embodiment, an example of the form in which the medical support program 94 is stored in the NVM 90 has been described. However, the technology of the present disclosure is not limited thereto. For example, the medical support program 94 may be stored in a portable non-transitory storage medium such as an SSD or a USB memory. The medical support program 94 stored in the non-transitory storage medium is installed in the computer 82 of the duodenoscope apparatus 12. The processor 76 performs the medical support process according to the medical support program 94.

In addition, the medical support program 94 may be stored in a storage device of, for example, another computer or a server that is connected to the duodenoscope apparatus 12 through a network. Then, the medical support program 94 may be downloaded and installed in the computer 82 in response to a request from the duodenoscope apparatus 12.

In addition, all of the medical support program 94 does not need to be stored in the storage device of, for example, another computer or the server connected to the duodenoscope apparatus 12 or the NVM 90, and a portion of the medical support program 94 may be stored therein.

The following various processors can be used as hardware resources for performing the medical support process. An example of the processor is a CPU which is a general-purpose processor that executes software, that is, a program, to function as the hardware resource performing the medical support process. In addition, an example of the processor is a dedicated electronic circuit which is a processor having a dedicated circuit configuration designed to perform a specific process, such as an FPGA, a PLD, or an ASIC. Any processor has a memory provided therein or connected thereto. Any processor uses the memory to perform the medical support process.

The hardware resource for performing the medical support process may be configured by one of the various processors or by a combination of two or more processors of the same type or different types (for example, a combination of a plurality of FPGAs or a combination of a CPU and an FPGA). Further, the hardware resource for performing the medical support process may be one processor.

A first example of the configuration in which the hardware resource is configured by one processor is an aspect in which one processor is configured by a combination of one or more CPUs and software and functions as the hardware resource for performing the medical support process. A second example of the configuration is an aspect in which a processor that implements the functions of the entire system including a plurality of hardware resources for performing the medical support process using one IC chip is used. A representative example of this aspect is an SoC. As described above, the medical support process is achieved using one or more of the various processors as the hardware resource.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The content described and illustrated above is a detailed description of portions related to the technology of the present disclosure and is only an example of the technology of the present disclosure. For example, the description of the configurations, functions, operations, and effects is the description of examples of the configurations, functions, operations, and effects of the portions related to the technology of the present disclosure. Therefore, it goes without saying that unnecessary portions may be deleted or new elements may be added or replaced in the content described and illustrated above, without departing from the gist of the technology of the present disclosure. In addition, the description of, for example, common technical knowledge that does not need to be particularly described to enable the implementation of the technology of the present disclosure is omitted in the content described and illustrated above in order to avoid confusion and to facilitate the understanding of the portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case in which the connection of three or more matters is expressed by “and/or”.

All of the documents, the patent applications, and the technical standards described in the specification are incorporated by reference herein to the same extent as each individual document, each patent application, and each technical standard is specifically and individually stated to be incorporated by reference.

What is claimed is:

1. A medical support device comprising:
a processor used for an endoscope apparatus including a camera that images a luminal organ and a treatment tool that is energized in a state of being in contact with a treatment target part included in the luminal organ to cut the treatment target part,
wherein the processor is configured to output permission and non-permission information indicating whether or not the energization is permitted, and
whether or not the energization is permitted is determined on the basis of a captured image that has been obtained by imaging the luminal organ with the camera and that includes the treatment tool.

2. The medical support device according to claim 1,
wherein the treatment tool has a conductor that comes into contact with the treatment target part, and
the conductor is included in the captured image.

3. The medical support device according to claim 2,
wherein the processor is configured to output the permission and non-permission information on the basis of an image in which the conductor and an insulating region insulating the conductor are included as the captured image.

4. The medical support device according to claim 1,
wherein the treatment tool has an insulating region, and
the energization is permitted in a case in which the insulating region is included in the captured image.

5. The medical support device according to claim 1,
wherein the treatment tool has an insulating region and a conductor that comes into contact with the treatment target part, and
the energization is permitted in a case in which the insulating region and the conductor are included in the captured image.

6. The medical support device according to claim 3,
wherein an indicator that is capable of specifying the insulating region is given to the insulating region.

7. The medical support device according to claim 1,
wherein an indicator is given to the treatment tool, and
the processor is configured to output the permission and non-permission information on the basis of an image in which the indicator is included as the captured image.

8. The medical support device according to claim 1,
wherein a process based on the captured image is performed as a process required to determine whether or not the energization is permitted in a case in which a first instruction has been received by a receiving device, and
the permission and non-permission information is output in a case in which a second instruction has been received by the receiving device.

9. The medical support device according to claim 1,
wherein whether or not the energization is permitted is determined on the basis of a size and/or position of the treatment tool included in the captured image.

10. The medical support device according to claim 1,
wherein the treatment tool has a conductor that comes into contact with the treatment target part, and
whether or not the energization is permitted is determined on the basis of a size and/or position of the conductor included in the captured image.

11. The medical support device according to claim 1,
wherein the treatment tool has an insulating region, and
whether or not the energization is permitted is determined on the basis of a size and/or position of the insulating region included in the captured image.

12. The medical support device according to claim 1,
wherein the endoscope apparatus has an insertion portion that is inserted into the luminal organ,
the camera is provided in the insertion portion,
the insertion portion has an opening and an insertion passage that leads to the opening,
the insertion passage has a conductive region,
the treatment tool protrudes from the opening through the insertion passage,
an insulating region is provided in the treatment tool, and
whether or not the energization is permitted is determined according to an aspect in which the treatment tool is included in the captured image.

13. The medical support device according to claim 1,
wherein the treatment tool includes a wire.

14. The medical support device according to claim 13,
wherein the treatment tool is a papillotomy knife including the wire.

15. The medical support device according to claim 13,
wherein the treatment tool is a snare including the wire.

16. The medical support device according to claim 13,
wherein the processor is configured to:
set an energized state in which the energization is performed on the wire on condition that a state of the wire included in the captured image is a treatable state in which the treatment target part is capable of being treated by performing the energization; and
set a non-energized state in which the energization is not performed on the wire on condition that the state of the wire included in the captured image is not the treatable state.

17. The medical support device according to claim 1,
wherein whether or not the energization is permitted is determined according to an aspect in which the treatment tool is included in the captured image.

18. The medical support device according to claim 1,
wherein the treatment tool has a conductor that comes into contact with the treatment target part, an insulating region, and at least one indicator, and
the conductor, the insulating region, and/or the at least one indicator are included in the captured image.

19. The medical support device according to claim 1,
wherein an object treated by the treatment tool is a duodenal papilla.

20. The medical support device according to claim 1,
wherein the output of the permission and non-permission information is displaying the permission and non-permission information on a screen.

21. The medical support device according to claim 1,
wherein the captured image is displayed on a screen.

22. An endoscope apparatus comprising:
the medical support device according to claim 1; and
the camera.

23. A medical support method used for an endoscope apparatus including a camera that images a luminal organ and a treatment tool that is energized in a state of being in contact with a treatment target part included in the luminal organ to cut the treatment target part, the medical support method comprising:
outputting permission and non-permission information indicating whether or not the energization is permitted,
wherein whether or not the energization is permitted is determined on the basis of a captured image that has been obtained by imaging the luminal organ with the camera and that includes the treatment tool.

24. A non-transitory computer-readable storage medium storing a program executable by a computer used for an endoscope apparatus including a camera that images a luminal organ and a treatment tool that is energized in a state of being in contact with a treatment target part included in the luminal organ to cut the treatment target part to perform a process comprising:
outputting permission and non-permission information indicating whether or not the energization is permitted,
wherein whether or not the energization is permitted is determined on the basis of a captured image that has been obtained by imaging the luminal organ with the camera and that includes the treatment tool.
